(19) United States
(12) Patent Application Publication
(10) Pub. No.: US 2016/0128947 A1
(43) Pub. Date: May 12, 2016

(54) BIOADHESIVE FILMS FOR LOCAL AND/OR SYSTEMIC DELIVERY

(71) Applicants: Jason Thomas MCCONVILLE, Albuquerque, NM (US); Javier O. MORALES, Universidad De Chile (CL); Alistair ROSS, Scotland (GB)

(72) Inventors: Jason Thomas McConville, Albuquerque, NM (US); Javier O. Morales, Universidad De Chile (CL); Alistair Ross, Clarkson, East Renfrewshire (GB)

(73) Assignee: STC. UNM, Albuquerque, NM (US)

(21) Appl. No.: 14/897,140

(22) PCT Filed: Mar. 15, 2013
(86) PCT No.: PCT/US2013/032490

§ 371 Date: Dec. 9, 2015

Related U.S. Application Data
(60) Provisional application No. 61/717,082, filed on Oct. 22, 2012.
(60) Provisional application No. 61/719,922, filed on Oct. 29, 2012.
(60) Provisional application No. 61/757,057, filed on Jan. 25, 2013.

(51) Int. Cl. A61K 9/70; B29B 13/00; A61K 38/28; A61K 9/00; A61K 31/522

(52) U.S. Cl. CPC A61K 9/7007; A61K 9/0021; A61K 31/522; A61K 9/006; A61K 38/28; B29B 13/00; B29K 2033/08

(57) ABSTRACT

Bioadhesive films suitable for topical, local, and systemic drug delivery and methods for making the same. The films may incorporate one or more polymeric layers that enable delivery of a specific, desired dosage, to a specific, desired location over a specific, desired time period.

### CROSS-REFERENCE TO RELATED APPLICATIONS

The following application claims benefit of U.S. Provisional Application Nos. 61/717,082, filed Oct. 22, 2012, 61/719,922, filed Oct. 29, 2012, and 61/757,017, filed Jan. 25, 2013, each of which is hereby incorporated by reference in its entirety.

### BACKGROUND

The development of films as bioadhesive dosage forms for buccal delivery of actives is a field that continues to grow due to unique characteristics that are advantageous for drug delivery (See e.g., Salamat-Miller et al., The use of bioadhesive polymers in buccal drug delivery. Adv Drug Deliv Rev. 2005; 57(11):1666-91; Sudhakar et al., Buccal bioadhesive drug delivery—a promising option for orally less efficient drugs. J Control Release. 2006; 114(1):15-40; Morales et al., Manufacture and characterization of bioadhesive buccal films. Eur J Pharm Biopharm. 2011; 77 (2):187-99.). In physical terms, films may be preferred over tablets due to size, flexibility, and comfort. As adhesive dosage forms, films can be formulated for a variety of delivery regimens as well providing the opportunity for locally treating diseases by direct application. The buccal route also offers interesting advantages over the oral route mainly for molecules that could be rendered inactive through the gastrointestinal tract, i.e., peptides and proteins. In addition, rapid absorption and peak concentration can be elicited through the venous system that drains from the cheek (See e.g., Shojaei A H. Buccal mucosa as a route for systemic drug delivery: a review. J Pharm Pharm Sci Publ Can Soc Pharm Sci Soc Can Sci Pharm. 1998; 1(1):15-30.)

As many advantages are offered with the buccal drug delivery method, only a handful of products have reached the market, and currently only two products for oral mucosal drug delivery have been successfully commercialized, and one further product has finished a phase 2 clinical study. BioDelivery Sciences International have used their BioErodible Bioadhesive (BEMA™) technology platform to develop Onsolis™, a fentanyl buccal soluble film indicated to be administered in the buccal mucosa for the management of breakthrough pain in patients with cancer (See, e.g., BioDelivery Sciences International, Onsolis™, http://www.bdsi.com/onsolis.php. (2010 Sep. 24).). The formulation contains the bioadhesive polymers carboxymethyl cellulose, hydroxyethyl cellulose, and polycarbophil, along with a backing layer to direct drug release towards the buccal mucosa. Using the same technology platform, BioDelivery Sciences International have completed a phase 2 clinical study for BEMA™ Buprenorphine with a significant improvement in the primary efficacy endpoint, SPID-8 (sum of pain intensity differences at 8 hours), compared to that exhibited by the placebo. The other commercialized film product is Suboxone™ Film, a buprenorphine and naloxone sublingual film. Using a polymeric matrix based on polyethylene oxide and hydropropylmethyl cellulose rapid dissolution and absorption are achieved [Reckitt Benckiser Pharmaceuticals Inc., Suboxone™ Sublingual Film (buprenorphine and naloxone), http://www.suboxone.com/hcp/suboxone_film/Default.aspx. (2010 Sep. 24).].

### SUMMARY

According to various embodiments the present disclosure provides bioadhesive films suitable for topical, local, and systemic drug delivery and methods for making the same. According to various embodiments the bioadhesive films may be designed to deliver drugs via a mucosal membrane. The films may include one or more polymeric layers which impart various characteristics and abilities to the films. According to some embodiments, the drug may be present in the film as part of a drug-coated particle. According to still further embodiments the drug-coated particle may have a diameter in the nano- or micro-size range.

### BRIEF DESCRIPTION OF THE DRAWINGS

FIG. 1 is a schematic illustration of an exemplary film according to the present disclosure in which a single layer provides both drug-delivery and bioadhesive properties

FIG. 2 is a schematic illustration of an exemplary dual-layer film according to the present disclosure including a drug delivery layer and a bioadhesive layer.

FIG. 3 is a schematic illustration of another exemplary dual-layer film according to the present disclosure wherein a first layer serves as a drug barrier which restrictions the direction of drug movement away from the second combined drug-delivery and bioadhesive layer.

FIG. 4 is a schematic illustration of another exemplary tri-layer film according to the present disclosure wherein the film employs separate drug barrier, drug-delivery, and bioadhesive layers.

FIG. 5 is a schematic illustration of yet another embodiment of the film of FIG. 3 wherein the drug barrier layer covers both the top and sides of the film, further restricting the direction of drug movement away from the second combined drug-delivery and muscoadhesive layer.

FIG. 6 is a schematic illustration of yet another embodiment of the film of FIG. 4 wherein the drug barrier layer covers both the top and sides of the film, further restricting the direction of drug movement away from the drug-delivery and muscoadhesive layers.

FIG. 7 is a schematic illustration of a method for forming biological-coated particles according to an embodiment of the present disclosure.

FIG. 8 is a schematic illustration of an exemplary apparatus for forming biological-coated particles according to an embodiment of the present disclosure.

FIG. 9 is a schematic illustration of the various layers of the mucosal tissue showing penetration levels of topically, locally, and systemically delivered drugs.

FIG. 10 is an SEM micrograph of protein-loaded submicron particles from SPH02.

FIG. 11 is an SEM micrograph of protein-loaded submicron particles from SPH07.

FIG. 12 is an SEM micrograph of a cross-section of film FPH01 obtained by freeze-fracture. The bar in FIGS. 14-17 represents 20 μm.

FIG. 13 is an SEM micrograph of a cross-section of film FPH03 obtained by freeze-fracture.

FIG. 14 is an SEM micrograph of a cross-section of film FPH04 obtained by freeze-fracture.

FIG. 15 is an SEM micrograph of a cross-section of film FPH05 obtained by freeze-fracture.

FIG. 16 is an SEM micrograph showing a closer up view of the cross section of FIG. 15. In FIGS. 16-19, the bar represents 5 μm.

FIG. 17 is a closer up view of the cross section of FIG. 16.

FIG. 18 is a closer up view of the cross section of FIG. 17.

FIG. 19 is a closer up view of the cross section of FIG. 18.

FIG. 20 is a graph showing bioadhesive properties MAF and WoA for Lys-containing films.

FIG. 21 shows Lys release profiles from particle-containing films FPH01 (♦), FPH02 (▪), FPH03 (▴), FPH04 ( ), FPH05 (□), and the control FPH06 ().

FIG. 22 is a graph showing Lys relative activity ( ) obtained from infinity release studies from film formulations.

FIG. 23 is a graph showing Ins yield after manufacture and stability after one month.

FIG. 24 is an SEM micrograph of a cross-section of ERL-Ins film obtained by freeze-fracture. The bar 20 represents μm.

FIG. 25 is an SEM micrograph of a cross-section of ERL-HPMC-Ins film obtained by freeze-fracture. The bar represents 20 μm.

FIG. 26 is an SEM micrograph of a cross-section of ERL-Ins film obtained by freeze-fracture. The bar represents 5 μm.

FIG. 27 is an SEM micrograph of a cross-section of ERL-HPMC-Ins film obtained by freeze-fracture. The bar represents 5 μm.

FIG. 28 is a graph showing the maximum adhesion force ( ) and (▪) Work of adhesion for Ins-containing films, C974P and PCP are shown as typical bioadhesive materials for comparison.

FIG. 29 is a graph showing Ins release studies for ERL-Ins (♦) and ERL-HPMC-Ins (▪) formulations.

### DETAILED DESCRIPTION

In general, the present disclosure provides bioadhesive films for location-specific delivery of pharmaceutical compositions or other biological of interest and methods for use and manufacturing the same. According to various embodiments, the methods described herein can be used to produce bioadhesive films containing releasable, bio-active macromolecules or other biologicals. The films produced by the methods demonstrate the desirable properties of bioadhesion, homogenous particle distribution, adequate physical-mechanical properties, high yield, and retention of macromolecule activity.

For the purposes of the present disclosure, it will be understood that the term “film” is intended to be inclusive and encompass membranes, strips, layered polymers, layered matrices, thin polymer matrices, patches, or the like and that unless specifically stated otherwise, the present disclosure contemplates the use of any or all of the above.

In general, as used herein, the term “biological” or “biological of interest” is intended to have broad scope and encompass any substance that is delivered to a patient via the films described herein. However, in the present disclosure, the terms biological, macromolecule, molecule, protein, pharmaceutical composition, drug, etc. may be used in various embodiments, examples, and when referring to various experiments. It should be understood that while each of these terms may have a unique and/or specific definition and scope, unless specific statements are made to the contrary, the use of one particular term, such as drug, protein, or molecule in a particular embodiment or example does not necessarily preclude the suitability of any other substance, such as a macromolecule, polypeptide, or nucleic acid, in the same or a similar embodiment or example.

Furthermore, it will be understood that for ease of description, the term “patient” is generally used to refer to an individual who is the intended end-user of the presently described film, i.e. the individual who receives a dosage of the biological of interest from the film. However, unless specifically stated, the term “patient” is not intended to limit the recipient to a human or to limit the intended use of the presently described films to the alleviation, amelioration, treatment, diagnosis, or cure of a condition, disease, or the like, nor should the use of this term be interpreted as intending to limit the “biological” or other substance being described as being limited to only those which can alleviate, ameliorate, treat, diagnose, or cure a condition, disease, or the like.

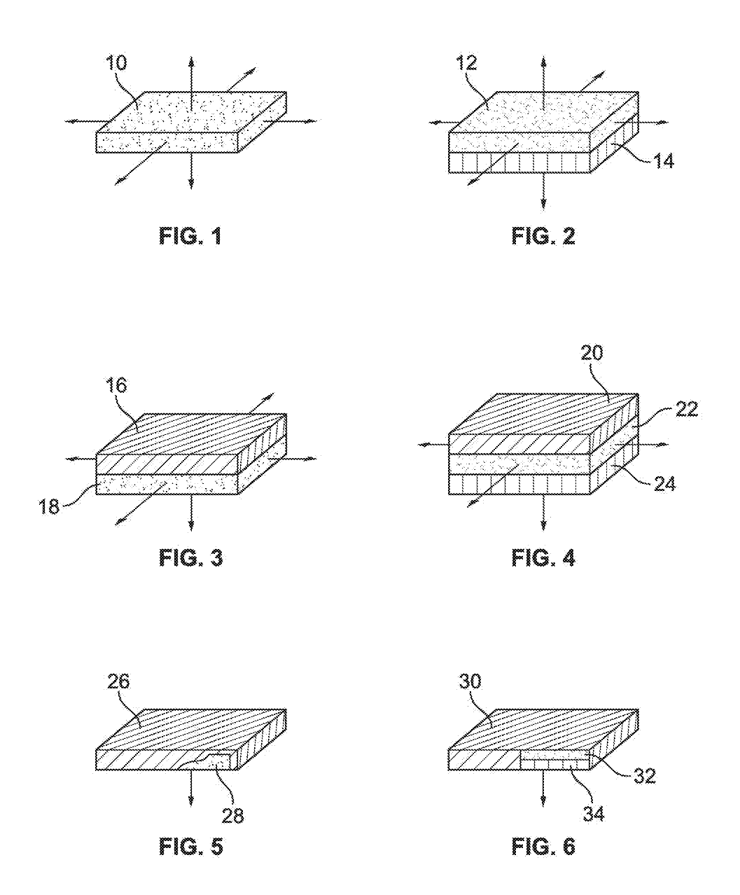

FIGS. 1-6 provide schematic illustrations of exemplary films according to the present disclosure. In general, the films of the present disclosure are formed from one or more layers of material with each layer providing one or more physical or chemical characteristic. While the films in the figures are shown as three-dimensional rectangular cubes formed from one or more rectangular layers, so that the disclosure can easily discuss and refer to various layers and elements of the films using commonly understood terms such as upper, lower, top, bottom, and sides, it will be understood that the shape of the films is not limited solely to one or more rectangular layers and that each layer and/or the film as a whole may be of any suitable regular or irregular 2- or 3-dimensional shape including, but not limited to circles, square, cylinders, cones, polyhedrons, polygons, crescents, etc. As explained in greater detail below, the films may be used in a variety of locations in the mouth or other areas of the body containing exposed mucosal layers and the shape of the films and layer(s) contained within the film may be informed based on the intended location of use for the film, for example, to increase comfort, for ease of placement or removal (if necessary), to increase or aid the film's ability to adhere, etc.

Furthermore, arrows are used in the figures to indicate the directionality of diffusion of drugs or other deliverables from the films. For ease of discussion, it will be assumed that the bottom of the film (as viewed in the drawings) will be placed against the muscosal layer of the patient, regardless of how the film would in fact be oriented in actual use (i.e. if the film was designed to attach to the roof of the mouth, the film would in fact be inverted relative to the orientation in the drawings).

According to various embodiments, the films of the present disclosure include at least the following three properties: bioadhesion, drug (or biological of interest) delivery, and specificity with respect to where and how the drug is delivered. Accordingly, FIGS. 1-6 demonstrate how various films can be designed to include specific combinations of layered materials in order to provide bioadhesion, drug delivery, and control over the location and directionality of drug delivery. The film shown in FIG. 1 is formed from a single bioadhesive drug layer 10, allowing the drug within the film to diffuse in all directions (as shown by the arrows). A similar result is achieved with the two-layered film shown in FIG. 2, which contains a non-drug containing bioadhesive layer 14 fused to a non-bioadhesive drug layer 12.

In contrast, the dual-layered film shown in FIG. 3 contains a bioadhesive drug layer 18 fused to drug barrier layer 16. Drug barrier layer 16 may be a layer formed from a material that is either impermeable or selectively permeable to the drug of interest. In general, it will be understood that regardless of the type of material used, the drug barrier layer 16 serves as a barrier to drug release, and thus limits the direction(s) in which the drugs can diffuse, as demonstrated by the lack of an upwardly pointing arrow in FIG. 3. An impermeable layer would generally be formed from a material that serves as a barrier to release or diffusion of all matter. An example of a material that might be used to form an impermeable layer would be wax. In contrast, a selectively permeable layer is designed to prevent diffusion or release of some, though not necessarily all matter. Typically, the selectively permeable layer would prevent release or diffusion of the drug of interest, but might allow other biologicals, molecules, substances etc., such as water, to be released, diffuse, or otherwise pass through. Examples of selectively permeable layers include insoluble celluloses, dialysis membranes, porous polycarbonate membranes and the like. For ease of discussion, the term “drug barrier layer” is intended to include layers formed from impermeable and selectively permeable materials.

The tri-layered example shown in FIG. 4 includes a drug barrier layer 20, a drug layer 22 and a bioadhesive layer 24. It is noted that in the examples shown in FIGS. 3 and 4, the drug is able to diffuse both through the bottom and sides of the film and that the drug barrier layer only prevents diffusion through the top of the film.

In FIGS. 5 and 6, the drug barrier layers 26 and 30, respectively, coat both the top and the sides of the film, preventing diffusion in all directions but downwards. The version shown in FIG. 5 includes a combined drug and bioadhesive layer 28, while the version shown in FIG. 6 shows a separate drug layer 32 and bioadhesive layer 34.

It will be readily understood that a single layer may perform more than one task. For example, the films shown in FIGS. 1, 3, and 5 contain a combined drug delivery/bioadhesive layer. In contrast, the films of FIGS. 2, 4, and 6 use multiple layers to accomplish each of these tasks. In addition to the combined drug delivery/bioadhesive layers described above, it will be understood that a single layer may be designed to include diffusion gradients or an active transport mechanic that reduces or eliminates the need for a separate drug barrier layer. Factors which may determine whether a film is designed to include layers capable of performing single or multiple tasks may be based on issues related to ease of manufacturing, access to appropriate materials, compatibility (or incompatibility) of materials to be included in a single layer, the desired shape of the final product and/or a combination of these or other factors.

In embodiments employing multiple layers, the various layers may be joined together by means of: electrostatic bonding, covalent bonds (e.g. thiolated bonds), crosslinking, hydrogen bonding, polymer entanglement, adhesion, cohesion, etc. In some embodiments, for example, the drug barrier layer may be a polymer-based coating that is poured over the preformed drug delivery/bioadhesive layer(s).

Furthermore, as explained in greater detail below, it will be understood that the films of the present disclosure may be designed with additional properties and/or able to deliver multiple types of biologicals and that such properties or abilities may or may not require the use of additional layers. Accordingly, the examples shown in FIGS. 1-6 are not intended to limit or dictate the maximum number of layers that may be included in a particular film.

We turn now more specifically to the first property identified above, bioadhesion. Bioadhesion is the ability of a material to adhere to mucosal membranes, which are located in the mouth (buccal mucosa) and also in the digestive, genital and urinary tracts. The films of the present disclosure are primarily intended for use with regard to buccal muscosa, but it will be appreciated that the films described herein will have uses related to other mucosal membranes and thus such uses are also contemplated.

The bioadhesive (or bioadhesive layers of the films) of the present disclosure are typically formed from a suitable biocompatible polymeric material. According to some embodiments, the polymers that are used as bioadhesives are predominantly hydrophilic polymers that swell and allow for chain interactions with the mucin molecules in the buccal mucosa. See e.g., Guo et al., Development of bioadhesive buccal patches. In. Bioadhesive Drug Delivery Systems: Fundamentals, Novel Approaches, and Development (Mathiowitz, E., Chickering, D. E., and Lehr, C. M., Eds.), pp 541-562 (1999), Marcel Dekker, Inc, New York. Examples of these swellable hydrophilic polymers include hydroxypropyl cellulose (HPC), hydroxypropylmethyl cellulose (HPMC), hydroxyethyl cellulose (HEC), sodium carboxymethyl cellulose (SCMC), poly(vinyl pyrrolidone) (PVP), and chitosan. However, in some cases hydrophobic polymers such as Eudragit® polymer grades can exhibit some bioadhesive properties when used alone, or in combination with other hydrophilic polymers. (See e.g., Wong et al., Formulation and evaluation of controlled release Eudragit buccal patches, International Journal of Pharmaceutics. (1999) 178, 11-22; Perumalet al., Formulation of monolayered films with drug and polymers of opposing solubilities, International Journal of Pharmaceutics. (2008) 358, 184-191; and Yehia et al., Fluconazole Bioadhesive Buccal Films: In Vitro/In Vivo Performance, Current Drug Delivery, (2009) 6, 17-27.) In fact, films containing propranolol hydrochloride, Eudragit RS100, and triethyl citrate as a plasticizer exhibited almost three times the bioadhesion force than that of films prepared with chitosan as the bioadhesive polymer (See Wong et al, above). A list of polymers that have been found to be suitable for use in the manufacture of buccal films, with additional descriptions and properties, is provided in Table 1 below.

TABLE 1
Bioadhesive polymers

| Bioadhesive | |
|---|---|
| polymer in | |
| films | Relevant properties and findings |
| Hydroxyethyl | Non-ionic polymer; High swelling properties and rapid |
| cellulose (HEC) | erosion; Low bioadhesive properties increased by the |
| | addition of SCMC; Zero order release kinetics of |
| | miconazole and chlorpheniramine |
| Hydroxypropyl | Non-ionic polymer; Increased swelling in ethylcellulose/ |
| cellulose (HPC) | HPC films; Moderate bioadhesive properties; Zero order |
| | release kinetics of lidocaine and clotrimazole associated |
| | with erosion; Square-root of time release kinetics |
| | of lidocaine |
| Hydroxypropyl- | Non-ionic polymer; Rapid swelling that plateaus; |
| methyl | Moderate bioadhesive properties; Initial burst followed |
| cellulose | by diffusion of nicotine hydrogen tartrate |
| (HPMC) | |
| Sodium | Anionic polymer; High swelling properties that does not |
| carboxymethyl | plateau; High bioadhesive properties; Zero order |
| cellulose | release of miconazole nitrate; Diffusion governed |
| (SCMC) | release of ibuprofen |
| Poly(vinyl | Non-ionic polymer; As film forming polymer exhibits |
| pyrrolidone) | non-fickian release of ketorolac and progesterone; Used |
| (PVP) | to tailor the release of propranolol and miconazole; |
| | High swelling properties; Used as coadjuvant to increase |
| | bioadhesion |
| Poly(vinyl- | Non-ionic polymer; Moderate swelling; and bioadhesive |
| alcohol) | properties; Anomalous release of miconazole |
| (PVA) | |
| Chitosan | Cationic polymer; High to moderate swelling and |
| | bioadhesive properties; Sustained release of miconazole |
| Alginate, | Anionic polymer; Rapid swelling and dissolution; |
| sodium | High bioadhesive properties |
| Agar | Poor and stable swelling properties |
| Carrageenan | Poor and stable swelling and moderate bioadhesive |
| type λ | properties |
| Acacia | Very poor bioadhesion |
| Guar gum | As an additive, conveyed moderate swelling and good |
| | bioadhesive properties, and anomalous non-fickian |
| | release of miconazole |
| Poly-L(lactide- | Micromatrices in buccal films to control the release |
| co-glycolide) | of ipriflavone |
| (PLGA) | |
| Polyacrilic | Rapid, high and stable swelling; High bioadhesive |
| acid, | properties; As a film forming polymer, conveyed |
| Carbopol ® | sustained release of buprenorphine; Used as an additive |
| | to tailor the release of propranolol |
| Polycarbophil | Non-ionic polymer; As an additive, conveyed moderate |
| | and stable swelling and high bioadhesive properties; |
| Poly(ethylene | Non-ionic polymer; High bioadhesion with high |
| oxide) | molecular weight; Zero order release kinetics of |
| | clotrimazole and tetrahidrocannabinol associated with |
| | erosion of the polymeric matrix |
| Poly(methacry- | Used as film former, exhibited very poor bioadhesive |
| lates) | properties and low swelling capability; The salt form |
| | has high bioadhesive properties. |

When selecting or designing a polymer for use in the films disclosed herein it will be understood that the mechanical properties of a film as a solid dosage form are of great importance since they account for the ability of the film to withstand various sources of stress. For example, the films need to be able withstand the stress imposed by manufacturing, handling, and administration. Additionally, films for buccal delivery need to be able to remain in contact with the mucosa for as long as the delivery of the active ingredient is ongoing. This involves mechanical stress originating from various mouth activities. Therefore, in some embodiments, the films of the present disclosure are preferred to exhibit a relatively high tensile strength (TS), elongation break (EB), and a low elastus modus (EM). In addition, regarding derived mechanical parameters, a relatively high TS/EM, Relative Surface Energy (RSE), and Toughness Index (TI) are desired. Methodologies for calculating and testing these properties and parameters are provided in Example I, below.

Furthermore, it will be understood that buccal delivery may be achieved by placement of the films at a variety of locations in patient's mouth including, for example, the cheek, hard palate (roof of the mouth), gingival region (gums), and lingual region (tongue), and, each of these regions has various and different textures, adhesive properties, mechanical properties, etc. Accordingly, it will be appreciated that it may be desirable to specifically design film for use in specific areas of the mouth and the intended area of use may therefore be another one of the many factors used to determine the appropriate polymer(s) to use. For example a low flexibility film may be suited to the hard palate but not to the buccal mucosa (cheek). It is possible to use many different polymers and tailor the mechanical properties by the use of additives (such as plasticizers) or affecting the manufacturing process to yield films of differing properties.

As specific non-limiting examples, for intended placement in the cheek, it may be desirable for the selected polymer to have a bioadhesive force of at least 100 mN and a work of adhesion of at least 50 μJ. Tensile strengths of greater than 0.5 N/mm², elongation at break of at least 50% and an elastic modulus of 0.4 N/mm²/% may be similarly desirable. For intended placement against the hard palate, it may be desirable for the polymer selected to have a bioadhesive force of at least 100 mN and a work of adhesion of at least 50 μJ. Tensile strengths of greater than 0.5 N/mm², elongation at break of at least 20% and an elastic modulus of 0.1 N/mm²/% may be similarly desirable. For intended placement in the gingival region, it may be desirable for the polymer selected to have a bioadhesive force of at least 100 mN and a work of adhesion of at least 50 μJ. Tensile strength of greater than 0.5 N/mm², elongation at break of at least 20% and an elastic modulus of 0.1 N/mm²/% may be similarly desirable. For intended placement on the tongue, it may be desirable for the polymer selected to have a bioadhesive force of at least 150 mN and a work of adhesion of at least 100 μJ. Tensile strengths of greater than 0.5 N/mm², elongation at break of at least 50% and an elastic modulus of 0.5 N/mm²/% may be similarly desirable.

Those of skill in the art will understand that the film thickness will be a product of the materials selected, the manufacturing process, and the particular design of the film. However, according to some embodiments, a film thickness of between 0.05 to 1 mm may be desired.

As stated above, according to various embodiments, the films of the present disclosure are used as a drug delivery vehicle. Furthermore, one goal of the presently described films, according to several embodiments, is the delivery of those drugs to the patient via penetration of the patient's mucosal membrane in a controlled and deliberate manner. Controlled and deliberate delivery typically requires some type of controlled release mechanism that differs from the passive and non-controlled release that occurs when a drug-containing film simply disintegrates upon contact with a wet surface (such as the mouth). (See e.g., the Triaminic® Cough Cold Thin Strips® or Allergy Thin Strips® which are commercial available from Novartis, Inc. (Basal, GE). Rapidly disintegrating strips such as these, while distributed to the patent in a strip form, are, in terms of the delivery mode, identical to orally disintegrating tablets or liquid medications (whereby the drug is already dissolved or dispersed) in that the drug itself is intended to be swallowed and then gains access to the patient's blood stream via the digestive system. These types of systems are primarily designed to be of use for patients with little access to water or for patients with varying degrees of dysphagia (difficulty swallowing especially in the young or elderly).

Therefore, unlike a passive, uncontrolled system wherein the local concentration of drug across the film is unimportant, a controlled, deliberate release system typically requires the drug to be precisely distributed throughout the drug containing layer. (Again it is emphasized that the drug containing layer may also serve as the bioadhesive or other layer within the film.) According to some embodiments, it is desirable for the drug concentration to be evenly distributed throughout the drug containing layer, so as to avoid areas of intense concentration, or clumping.

Accordingly, the present disclosure provides a mechanism for distributing a biological of interest, such as a drug or pharmaceutical composition, throughout a polymeric layer. As demonstrated by the results in the Examples section below, providing the biological of interest in the form of a biological-coated particle enables the manufacture of films having a relatively even distribution of the biological throughout the entire drug layer. Therefore, according to an embodiment of the present disclosure, the drugs or biological of interest is present in one or more layers of the film in the form of biological-coated particles imbedded in a polymeric film.

Furthermore, example II below demonstrates that under some circumstances, the presence of agglomerates due to increased biological loading changes the release kinetics of the film from a diffusion controlled mechanism to a first order mass balance. However, this effect is countered when the particles are limited to the submicron or nano-size range, as the potential for aggregation of the particles is reduced or eliminated. Accordingly, it may be desirable for the particles embedded in the presently described films to be in a submicron or nano size range.

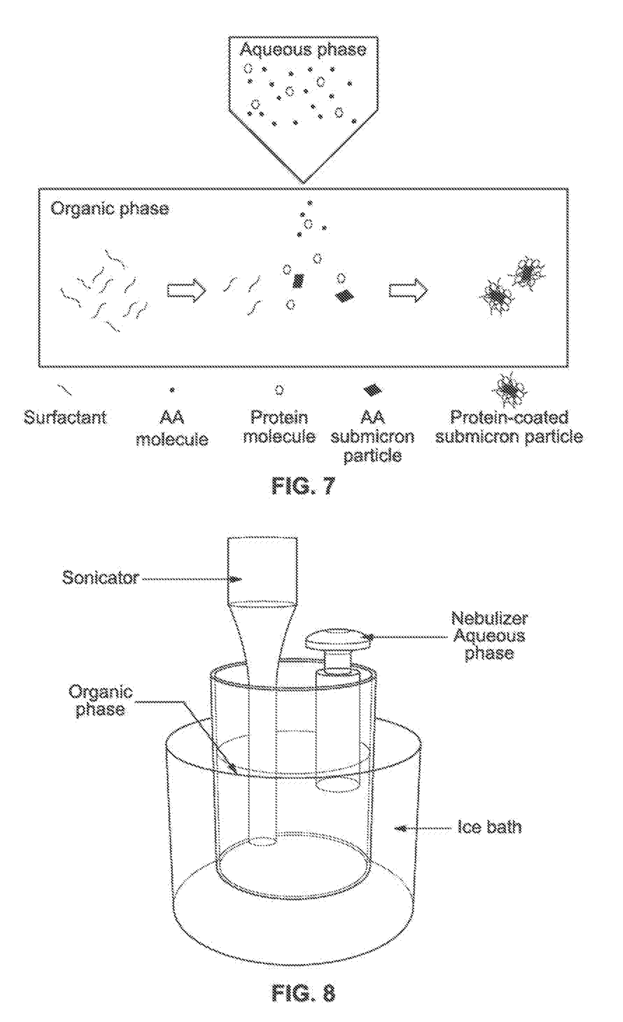

While it will be understood that any suitable nano or micro-sized biological coated particles could be used, in some embodiments it may be desirable to manufacture biological-coated particles in the sub-micron to nano-size range for inclusion in the films of the present disclosure. One suitable method of manufacturing biological-coated particles utilizes an antisolvent co-precipitation methodology, which combines the use of high energy mixing by means of a sonicator, stabilizing surfactants in the organic phase, and increased surface area for addition of the aqueous phase by means of nebulization in order to produce submicron sized and nanosized batches of particles coated with biologicals. Specific examples of the use of the antisolvent co-precipitation method to product lysozyme (Lys) loaded D,L-valine (Val) and insulin-coated submicron particles are provided in the Examples section, below. However, in general, the antisolvent precipitation process involves solubilizing the molecule of interest in a suitable solvent and then adding droplets of this solution to a miscible antisolvent in order to trigger precipitation. For example, as shown in FIG. 7, an aqueous solution containing the biological of interest (depicted as a protein molecule) and a co-precipitant (depicted as an amino acid molecule), which acts as a seed for the final precipitated particles, are prepared with the desired concentration of both ingredients. Droplets of this solution are then added to an antisolvent organic phase that is completely miscible with the aqueous phase but in which there is no solubility for either the biological of interest or the core forming co-precipitant. During addition of the aqueous phase, high energy mixing is provided. If an improvement in stability is needed, the protein molecule can be immobilized in its native form after precipitation. In this case, an aqueous solution containing the protein/peptide and seed co-precipitant is initially prepared and admixed with an organic water-miscible solvent (e.g. 2-propanol), which can optionally contain a surfactant.

Those of skill in the art will recognize that the selection of the solvent and antisolvent will depend largely on the properties of the molecule of interest and core material being used. In the Examples below, the molecule of interest was a protein and the core molecules were amino acid (selected due to their chemical compatibility with proteins.) In these conditions, an antisolvent such as Isopropyl alcohol (IPA) may be used. Alternative suitable antisolvents include but are not limited to alcohols such as methanol; ethanol; propan-1-ol; aldehydes or ketones such as acetone, esters such as ethyl lactate, ethers such as tetrahydrofuran, diols such as 2-methyl-2,4-pentanediol, 1,5-pentane diol, and various size polyethylene glycol (PEGS) and polyols; or any combination or mixture thereof. It is noted that our experiments showed that acetone and ethanol were not able to trigger precipitation of the amino acid particles under the specific conditions used. However, there is every reason to believe these would likely be suitable antisolvents for other materials and/or under other conditions.

As indicated above, amino acids are able to act as the co-precipitant core material. Other suitable core materials include but are not limited to organic and inorganic salts, buffer components, water soluble small-molecule drugs, sugars, sugar alcohols, zwitterions, peptides, inert seed materials (e.g. silica), colloidal seed materials, fatty acids, monomers, polymers. (See e.g., Nikolic et al., Self-assembly of nanoparticles on the surface of ionic crystals: Structural properties, Surface Science. (2007) 601, 2730-2734 and Murdan et al. Immobilisation of vaccines onto micro-crystals for enhanced thermal stability, International Journal of Pharmaceutics. (2005) 296, 117-121.)

Any suitable mechanism for adding droplets of the aqueous solution to the antisolvent may be used. For example, addition of the aqueous solution may be facilitated by use of a syringe pump to control the rate of delivery of the aqueous solution in a drop-wise fashion. As another example, droplets of the aqueous solution may be added by use of a nebulizer. Use of a nebulizer has the advantage of providing droplets in a very narrow and specific size range (for example in a range of 1-5 μm), thereby allowing for the formation of a relatively monodisperse population of particles in the low micron- to nano-size range.

A variety of mixing methods may be used including magnetic stirring, homogenization, and sonication. Sonication is a suitable method for a variety of embodiments, as it is effective in providing high energy mixing and small particle sizes. For example, the duration, intensity, and lapse length of sonication, both during and after addition of the aqueous phase to the antisolvent, were found to have an impact on particle size. In general, longer times, low intensity, and short duration of lapses demonstrated a high level of control for particle size and resulted in smaller particles.

Additional synthesis factors that may affect particle size include: the presence of surfactants in the aqueous phase; the volume of aqueous solution; particle shape; and loading of the biological of interest.

As stated above, according to various embodiments, the biological of interest, which may be, but need not necessarily take the form of, the biological-loaded micro- or nano-particles described above, is embedded into or otherwise releasably contained within a polymeric material to form the biological-containing layers of the present disclosure. Suitable polymeric materials include the bioadhesive polymers described above. However, as shown in FIGS. 2, 4, and 6, the drug-containing layer may not be bioadhesive.

It will be appreciated that the biological-containing polymer layer may be selected and/or designed to exhibit a variety of properties which can be exploited for use in the biological-delivery film of the present disclosure. For example, the polymer layer may be selected or designed to have a specific release rate for the biological of interest. This may be determined by factors such as: the polymer's rate of diffusion, density, swellability, rate of degradation, hydrophilicity, hydrophobicity, degree of crosslinking, degree of polymer substitution, molecular weight, ionization state, pKa of functional groups, etc. Additional information on the mechanisms by which polymers can control the release of an active ingredient may be found, for example, in Morales et al., Manufacture and characterization of bioadhesive buccal films, European Journal of Pharmaceutics and Biopharmaceutics. (2011) 77, 187-199; Dixit et al., Oral film technology: Overview and future potential, Journal of Controlled Release. (2009) 139, 94-107; and McQuinn et al., Oral transmucosal delivery of melatonin. In. Drug delivery to the oral cavity: molecules to market (Ghosh, T. K., and Pfister, W. R., Eds.) (2005), Marcel Dekker Inc, New York.

Furthermore, the films of the present disclosure may be designed to deliver one or more drugs at one or more delivery rates by providing multiple drug-containing layers containing different drugs and/or different release rates. These multiple drug-containing layers may be oriented relative to each other in any suitable or desired manner including completely or partially stacked one on top of the other, completely or partially side-by-side or in some other over-lapping, or non-overlapping manner.

As stated above, the films of the present disclosure are capable of delivering the biological of interest with a great deal of specificity with respect to where and how the drug is delivered. As shown in FIGS. 3-6, one mechanism for providing this specificity is the use of a drug barrier layer.

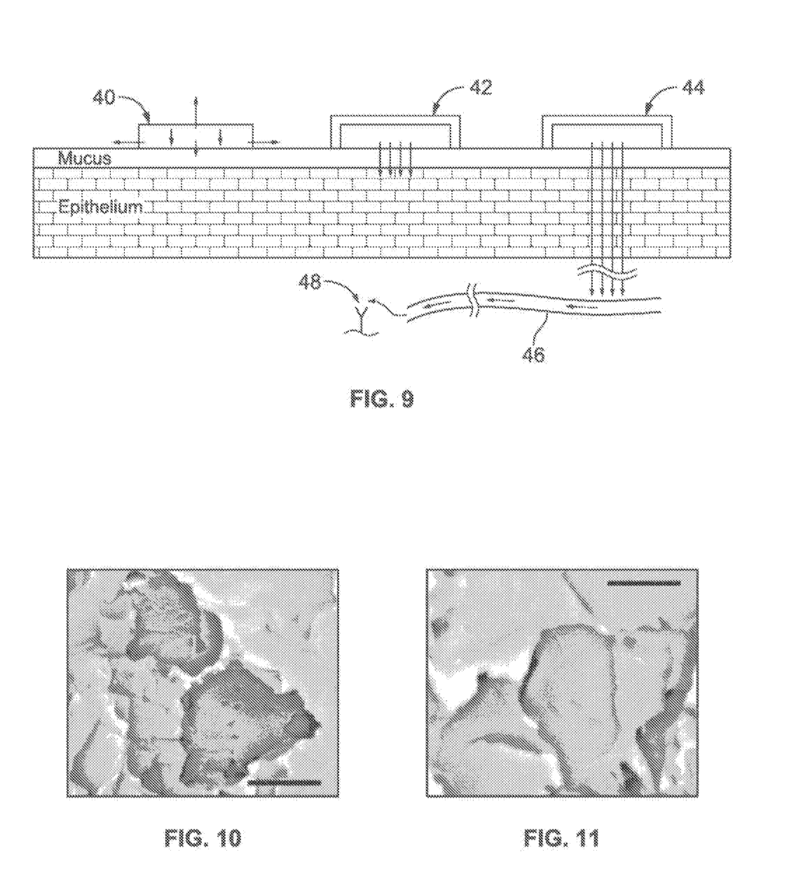

However, specificity is not limited to simply the directionality of the drug delivery, but may also be determined based on the intended target of the drug. FIG. 9 depicts the various layers of the buccal mucosa through which it may or may not be desirable for a given drug to permeate. As shown in FIG. 9, topical drug films, such as that shown at 40, are typically intended to deliver a drug such that it will affect only the surface or the upper most layers of the buccal mucosa. As shown, because the intended delivery area is the surface or upper layers, topical drug films may permit diffusion or drug delivery in multiple directions. Locally acting drug films, such as that shown at 42 may function to deliver drugs that are able treat local pain or ailments. Systemically active drug films, such as that shown at 44 are typically able to deliver a drug such that it can penetrate all layers of the (oral) epithelium via the (buccal) mucosa through to the vasculature where absorption can occur and the drug is able to enter the patient's blood stream 46 for ultimate efficacy at a suitable receptor 48 located elsewhere in the body.

Films containing drugs intended for topical delivery may be physically designed to be partially or entirely placed over or on top of the intended deliver area. A drug barrier layer may or may not be incorporated on the top and sides of the film. Furthermore, if the targeted area if very small, a portion of the bottom of the film may contain a drug barrier layer. Examples of topical films include films intended to treat or ameliorate oral pain from burns, sores, dental work, gum tenderness, teething, etc.

Films containing drugs intended for local delivery will frequently contain a drug barrier layer on the top of the film, and may or may not contain a drug barrier layer on the sides of the film. The rate and target area of delivery may be largely dependent on particular polymer or combinations of polymers used, the use of excipients, and the concentration of the drug (and its specific physicochemical properties) in the drug delivery layer. Additionally, the treatment regimen (i.e. once or twice daily etc.) or the modality of treatment (e.g. rapid or extended release delivery), as well as the type of disease are parameters that may be considered in determining the configuration and construction of the film.

Similar to local delivery films, films containing drugs intended for systemic delivery will frequently contain a drug barrier layer on the top of the film, and may or may not contain a drug barrier layer on the sides of the film. Furthermore, systemic drugs may be delivered as a single bolus delivery, in an extended time-dependent manner, or as a combination thereof. As with local delivery films, the rate and target area of delivery may be largely dependent on particular polymer or combinations of polymers used, the use of excipients, and the concentration of the drug (and its specific physicochemical properties) in the drug delivery layer. Additionally, the treatment regimen (i.e. once or twice daily etc.) or the modality of treatment (e.g. rapid or extended release delivery), as well as the type of disease are all parameters that may be considered in determining the configuration and construction of the film.

Systemic delivery films may also incorporate permeation enhancers into their design. Permeation enhancers enhance or enable various intra or extra cellular transport mechanisms, allowing a drug or other biological of interest to travel through cellular layers that they would normally have difficulty navigating. Examples of permeation enhancers include: surfactants such as sodium dodecyl sulfate and sodium lauryl sulfate, which act through lipid extraction from the mucosa; bile salts such as sodium glycocholate, sodium taurocholate, sodium glycodeoxycholate, sodium taurodeoxycholate, and sodium deoxycholate which also act through lipid extraction from the mucosa; fatty acids such as oleic acid, eicosapentaenoic acid and docosahexaenoic acid, which increase the fluidity of intercellular lipids; ethanol which disrupts the arrangement of intercellular lipids; and chitosan, which both increases the retention time of a drug in contact with the mucosa and disrupts the intercellular lipid organization. These (or other) permeation enhancers may be incorporated into any suitable layer of the film including the drug-containing layer, bioadhesive layer, or other layer. Examples of systemic delivery films include insulin-loaded films which are configured to deliver constant low-dosage of insulin in order to maintain a diabetic patient's basal insulin levels, in place of a pump for example. In this case the insulin film could be replaced at a regular rate, for example, daily. Permeation enhancers could also be physical (e.g. biodegradable microneedles containing drug as a solid dispersion, or nanoencapsulated) to enable rapid transfer across the various tissues of the oral cavity.

Examples of diseases that might be treated or managed by systemic delivery of drugs via the films disclosed herein include diabetes (either bolus or extended delivery or a combination thereof), cardiovascular disease (bolus dose of drug), and/or antibiotic therapy (multiple bolus dose delivery or extended release to maintain systemic levels of drug). By altering various factors such as drug barrier layers, drug concentration, and permeation enhancers, the films of the present disclosure can be designed for topical, local, and/or systemic drug delivery, combinations thereof, or anything in between.

The various layers and films of the present disclosure may be manufactured using any suitable technique. Furthermore, individual layers may be manufactured separately and then assembled together or some or all layers may be manufactured together in a single process.

Examples of suitable manufacturing methods for the various polymeric layers described herein include solvent casting and hot melt extrusion. According to one embodiment, one or more layers of the presently described films may be formed using a solvent casting process. Film casting is a widely used manufacturing process for making films. The process generally includes at least six steps: preparation of the casting solution; deaeration of the solution; transfer the appropriate volume of solution into a mold; drying the casting solution; cutting the final dosage form to contain the desired amount of drug; and packaging. For example, in order to produce the drug-containing layers described herein, a casting solution including the biological-coated sub-micron sized particles such as those described above and precursors for a bioadhesive or non-muscoadhesive polymer film as described above may be prepared and a suitable film cast therefrom.

An alternative method of producing the films described herein is hot melt extrusion. In hot melt extrusion, a blend of pharmaceutical ingredients is molten and then forced through an orifice (the die) to yield a more homogeneous material in different shapes, such as granules, tablets, or films. Hot-melt extrusion has been used for the manufacture of controlled release matrix tablets, pellets, and granules; as well as orally disintegrating films.

It will be appreciated that the films may include additional layers or other excipients to control for different properties of the films. Examples include, but are not limited to, film forming polymers, plasticizers, taste masking or sweetening ingredients, and stabilizers. As a specific example and as discussed in greater detail in the Examples section, the release of Lys can be controlled or tuned with the use of HPMC as a water-swellable and soluble material in the form.

As demonstrated in greater detail in the Examples section below, the methods described herein produce films having homogenously distributed particles with a narrow particle size distribution, high yields, and high stability.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims.

All patents and publications referenced below and/or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

References: Additional information and disclosure may be obtained by review of the Examples below.

### EXAMPLES

### Example 1

### Water-Swellable Polymethacrylate Bioadhesive Buccal Films Containing Caffeine Particles

In this investigation we sought to evaluate systematically the performance of Eudragit RS (ERS) and Eudragit (ERL) as bioadhesive polymers to be suitable for the delivery of the water soluble model drug caffeine.

For the fabrication of films, polymers were firstly dissolved in an acetone:isopropanol (4:6 ratio) solvent system and then 10% w/w triethyl citrate was added as plasticizer. Increasing quantities of caffeine were added to yield solutions containing 1, 2, 3, 4, or 5% w/w caffeine. Films made of both, ERS and ERL polymers were obtained for each concentration. These solutions were casted on PTFE plates and let to dry overnight at 40° C. to yield the final product. To compare with conventional bioadhesive materials, films containing C974P and PCP were manufactured similarly. Adequate amounts of the polymers were dissolved in ethanol and then cast in the same fashion as described above.

SEM images shown revealed that increasing the concentration of caffeine in both ERS and ERL films leads to an increasing appearance of agglomerates in cross sections of films obtained by freeze fracture. A survey of cross sections reveals that the use of ERS leads to a higher quantity and larger size of these agglomerates at similar concentrations of caffeine compared to those seen in ERL films.

From stress vs. strain curves, TS, EB, and EM were obtained and the derived magnitudes of TS/EM, RSE, and TI were computed for each sample and are summarized in Tables 2 and 3.

TABLE 2
Mechanical properties of formulations from ERS and ERL series. Values
are represented as average and standard deviation in parenthesis.

| | Tensile Strength/ | Elongation at | Elastic Modulus/ |
|---|---|---|---|
| Formulation | N/mm² | Break/% | N/mm²/% |
| ERS01 | 5.71 (1.72)^{a} | 142.19 (35.46)^{ab} | 1.19 (0.46) |
| ERS02 | 3.62 (0.61) | 162.40 (44.06)^{cde} | 1.18 (0.04) |
| ERS03 | 4.16 (0.86) | 82.88 (20.74)^{c} | 1.33 (0.26) |
| ERS04 | 4.04 (0.83) | 35.30 (6.24)^{ad} | 1.27 (0.36) |
| ERS05 | 2.48 (0.14)^{a} | 35.82 (17.99)^{bc} | 0.80 (0.09) |
| ERL01 | 1.51 (0.19)^{ab} | 233.04 (23.85)^{a} | 0.43 (0.05)^{ab} |
| ERL02 | 1.17 (0.13)^{acd} | 262.21 (34.06)^{b} | 0.34 (0.05) |
| ERL03 | 0.75 (0.06)^{bcef} | 275.23 (35.84)^{cd} | 0.24 (0.03)^{a} |
| ERL04 | 1.26 (0.17)^{c} | 221.83 (30.38)^{ce} | 0.43 (0.04) |
| ERL05 | 1.51 (0.14)^{df} | 93.41 (9.65)^{abde} | 0.63 (0.07)^{b} |

^{a, b, c, d, e, f}Among parameters and between series of formulations, statistically significant differences are paired by the same letters (p < 0.01).

TABLE 3
Derived mechanical parameters calculated from conventional
mechanical properties derived a Stress vs. Strain plot. Values are
represented as average and standard deviation in parenthesis.

| | | Relative | |
|---|---|---|---|
| | | Surface | Toughness |
| | TS:EM | Energy | index |
| Formulation | %⁻¹ | N/mm² · % | N/mm² · % |
| ERS01 | 4.98 (0.84)^{a} | 13.90 (3.23)^{abcd} | 515.88 (38.39)^{abc} |
| ERS02 | 3.23 (0.53)^{a} | 6.25 (1.95)^{a} | 391.16 (116.40)^{def} |
| ERS03 | 3.14 (0.46) | 6.60 (1.97)^{b} | 222.07 (5.69)^{adg} |
| ERS04 | 3.35 (1.14) | 6.91 (3.27)^{c} | 96.82 (34.12)^{bc} |
| ERS05 | 3.16 (0.54) | 3.95 (0.91)^{d} | 58.53 (28.48)^{cfg} |
| ERL01 | 3.31 (0.65) | 2.38 (0.71) | 233.04 (27.10)^{ab} |
| ERL02 | 3.29 (0.23) | 1.80 (0.07) | 204.80 (33.14)^{cd} |
| ERL03 | 3.14 (0.71) | 1.17 (0.37) | 136.80 (8.07)^{ac} |
| ERL04 | 2.94 (0.57) | 1.88 (0.63) | 186.94 (41.36)^{c} |
| ERL05 | 2.44 (0.39) | 1.86 (0.45) | 94.53 (15.45)^{bde} |

^{a, b, c, d, e, f}Among parameters and between series of formulations, statistically significant differences are paired by the same letters (p < 0.01).

We found that the bioadhesive properties of ERS are very limited under our specific test conditions. However, ERL is highly bioadhesive under the test conditions utilized here. Our studies revealed that the extent of bioadhesion found with ERL is comparable to that of typical bioadhesive materials, namely C974P and PCP. It was found however that the WoA was about 80%, significantly higher than conventional bioadhesive materials (118.9 vs. 23.9 and 17.4 μJ), demonstrating that a highly swellable polymer, such as ERL, regardless of being water-insoluble, can elicit strong bioadhesiveness based on its capacity for entanglement. The various films in the ERL series exhibit high WoA and high MAF when a drug is solubilized in the polymer or small micron size agglomerates are found (ERL01-ERL04)

### Example II

### Protein-Containing Submicron Particles Embedded in Films for Buccal Delivery

Bioadhesive films intended for the buccal delivery of macromolecules were developed using Lys as model to yield high enzyme activity submicron particles. The particle manufacturing process was based on antisolvent co-precipitation. Briefly, the co-precipitant Val and the amount of Lys to be precipitated were dissolved in one of the buffers and solutions studied (Table 4). First, Val was dissolved in the aqueous phase at a concentration of 61.2 mg/mL (or 90% of its saturation concentration) and then Lys was dissolved in this solution to yield a protein content of 40% w/w based on solid content. By means of an Aeroneb Pro® vibrating mesh nebulizer, the aqueous phase was then added to the antisolvent organic phase. A 0.008 mM Span60 IPA solution was utilized as the antisolvent. Finally, during the addition of the aqueous phase high energy mixing is provided by means of a Branson Sonifier 450 probe sonicator. After addition of the total volume of aqueous phase sonication is maintained for 20 more minutes.

TABLE 4
Formulations prepared to study the effect of pH
in the manufacturing process of Lys PCNP.

| Formulation | Protein model | Buffer/solution | pH |
|---|---|---|---|
| SPH01 | Lys | N Phthalate | 5.4 |
| SPH02 | Lys | Phosphate | 6.8 |
| SPH03 | Lys | Borate | 10 |
| SPH04 | Lys | NaOH | 13 |

Casting solutions were prepared by combining two organic solutions and cast overnight in PTFE molds. Acetone was used to dissolve or suspend the polymer combinations as depicted in Table 5. This solution was combined in a 4:6 acetone to IPA ratio with suitable amounts of SPH02 (pH 6.8) of Lys-containing IPA (for the control formulation, FPH06) to yield the final casting solution. After 24 hours, films were peeled off and stored in aluminum foil sachets in a dessicator until characterization.

TABLE 5
Film formulation compositions (as % w/w) that were
studied to investigate drug release and
uniformity of films containing SPH02.

| Formulation | Eudragit RL | Eudragit RS | HPMC | TEC | IPA solution |
|---|---|---|---|---|---|
| FPH01 | 90 | — | — | 10 | SPH02 |
| FPH02 | 73 | — | 17 | 10 | SPH02 |
| FPH03 | 64 | — | 26 | 10 | SPH02 |
| FPH04 | 45 | — | 45 | 10 | SPH02 |
| FPH05 | — | 90 | — | 10 | SPH02 |
| FPH06 | 90 | — | — | 10 | Lys* |

*Unprocessed Lys was dispersed in IPA to prepare the control formulation FPH06.

We investigated the effect of pH in the aqueous phase containing Lys over particle size, yield, and stability in connection with the antisolvent co-precipitation method among others.

FIGS. 10 and 11 are SEM micrographs of protein-loaded submicron particles from selected formulations SPH02 (FIG. 10) and SPH07 (FIG. 11). The bar represents 1 μm.

A narrow particle submicron size distribution was obtained at optimized conditions. SPH02 at pH 6.8 was found to be the best condition for the precipitation of Lys. This formulation yielded very small particle sizes (347.2±16.9 nm), adequate PdI, and low variability (Table).

TABLE 6
Particle size reported as z-average, polydispersity index and
zeta potential of Lys formulations. Results are represented
as the mean and standard deviation in parenthesis.

| Formulation | Z-average/nm* | Polidispersity index* | Zeta Potential/mV |
|---|---|---|---|
| SPH01 | —** | —** | —** |
| SPH02 | 347.2 (16.9) | 0.36 (0.02)ⁱ | 21.9 (3.7)^{a} |
| SPH03 | 1384.0 (152.7) | 0.28 (0.13)ii | 18.3 (2.0)^{a} |
| SPH04 | 1220.2 (426.6) | 0.43 (0.13) | 10.1 (1.2) |

*Among parameters, all differences were statistically significant (p < 0.05).
^{a, b}Among parameters, non-significant differences are indicated in pairs of letters, all other differences are significant (p < 0.05).
**co-precipitant and Lys did not dissolve at pH 5.4.

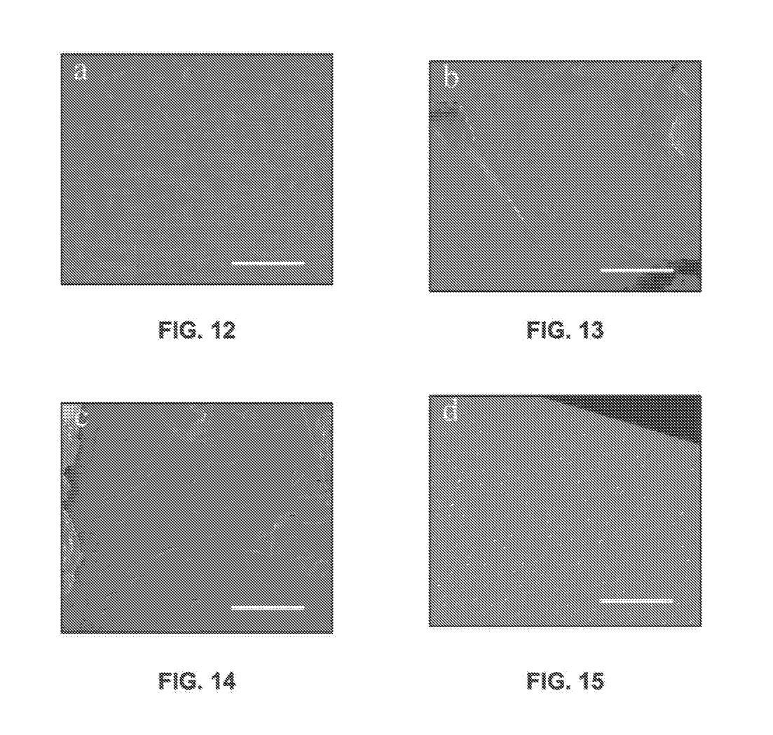

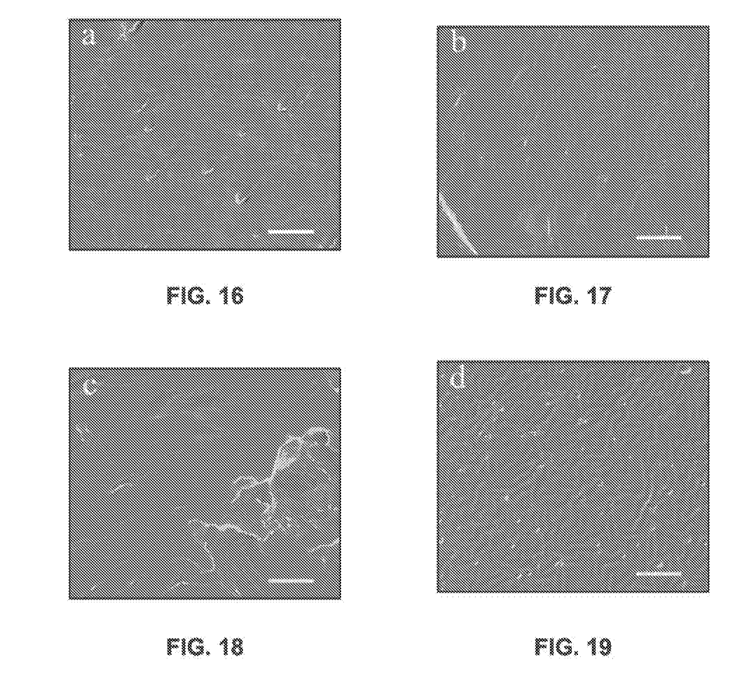

Regardless of the pH, excellent Lys yield and stability was achieved. With yields in the range of 70.5-73.4 (no statistical differences found, p<0.05) and remaining relative activity in the range of 91.4-101.1% we corroborated that the method of manufacture of submicron particles by the antisolvent co-precipitation method is successful in rendering functional particles. This also indicates that the pH of the buffer solution containing Lys before manufacture had little effect on the resulting stability after manufacture. Films were successfully manufactured and their surface appeared homogeneous to the eye. An SEM observation of cross sections of selected film formulations obtained by freeze fracture reveals a uniform distribution of the flake-like particles throughout the polymeric matrix (FIGS. 12-15). A closer look (FIGS. 16-19) shows that mostly individual particles are separately enclosed resulting in high drug content uniformity in the films. Agglomerates of HPMC can also be observed homogeneously distributed under the SEM, indicating the composite nature of the film.

Bioadhesion and Mechanical Properties of Lys-Containing Films

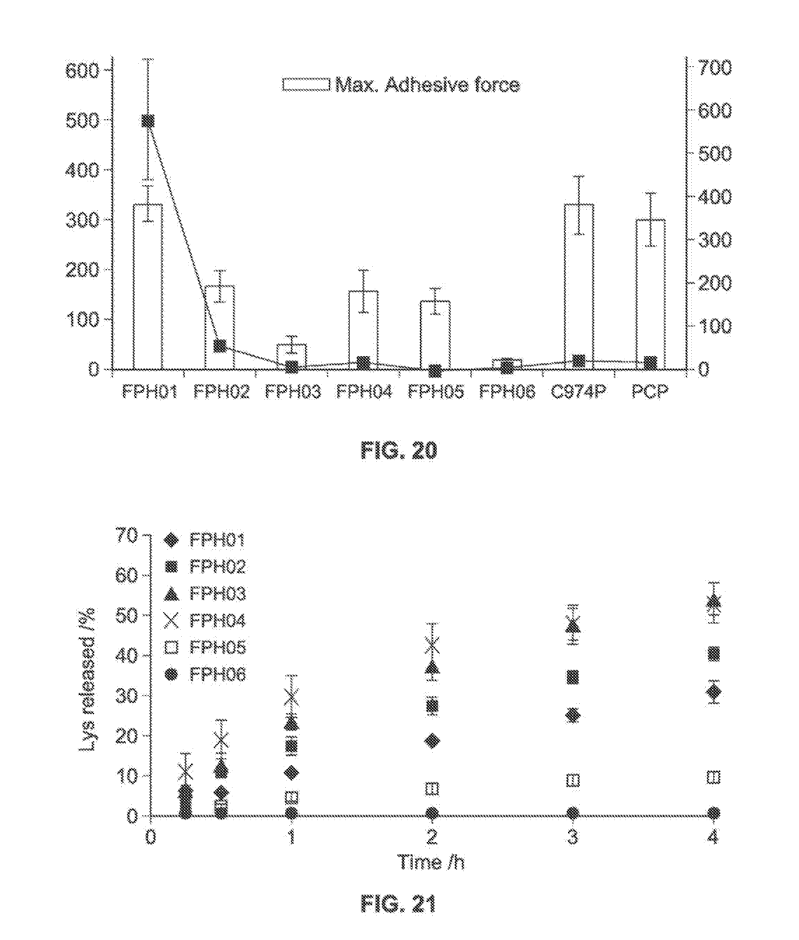

FIG. 20 shows the bioadhesive properties ( ) MAF and (▪) WoA for Lys-containing films. The same values for conventional bioadhesive polymers such as C974P and PCP are depicted for comparing the performance of films developed here. a-h: Non-significant differences among MAF are indicated in pairs of letters. Only WoA of FPH01 was significantly different from other formulations (p<0.05). All other values were not statistically different (p>0.05).

TABLE 7
Mechanical properties for Lys-containing films. Results are
represented as the mean and standard deviation in parenthesis.

| | Tensile Strength/ | Elongation at | Elastic modulus/ |
|---|---|---|---|
| Formulation | N/mm2 | break/% | N/mm2/% |
| FPH01 | 1.653 (0.160)^{a} | 179.9 (26.7)^{a} | 0.318 (0.110)^{a,b} |
| FPH02 | 2.783 (0.133) | 50.0 (11.0)^{b,c} | 0.831 (0.048) |
| FPH03 | 5.169 (0.462)^{b} | 25.6 (6.5)^{b,d} | 1.554 (0.191) |
| FPH04 | 5.005 (1.464)^{b} | 18.0 (4.0)^{c,d} | 1.228 (0.129) |
| FPH05 | 0.580 (0.075)^{c} | 233.6 (43.9)^{a} | 0.153 (0.038)^{a} |
| FPH06 | 1.273 (0.124)^{a,c} | 124.7 (12.9) | 0.465 (0.093)^{b} |

^{a-d}Among parameters, non-significant differences are indicated in pairs of letters.

TABLE 8
Derived mechanical parameters calculated from conventional
mechanical properties derived from a stress vs. strain plot. Results
are represented as the mean and standard deviation in parenthesis.

| | | Relative | |
|---|---|---|---|
| | | Surface | Toughness |
| | TS:EM | Energy | index |
| Formulation | %⁻¹ | N/mm² · % | N/mm² · % |
| FPH01 | 5.69 (1.94)^{i,ii,iii} | 4.59 (1.06)^{a} | 216.51 (21.32)^{i,ii,iii,iv,v} |
| FPH02 | 3.36 (0.32)ⁱ | 4.69 (0.63)^{a} | 92.08 (15.55)ⁱ |
| FPH03 | 3.34 (0.22)ⁱⁱ | 8.62 (0.77)^{b} | 88.10 (22.67)ⁱⁱ |
| FPH04 | 4.11 (0.50) | 10.32 (1.93)^{b} | 60.32 (15.70)^{iii,vi} |
| FPH05 | 3.88 (0.50) | 1.11 (0.08)^{c} | 89.91 (17.96)^{iv} |
| FPH06 | 2.77 (0.29)ⁱⁱⁱ | 1.75 (0.08)^{c} | 106.33 (20.12)^{v,vi} |

^{i-vi}Among parameters, statistically significant differences indicated in pairs of roman numerals (p < 0.05).
^{a-c}Among parameters, non-significant differences are indicated in pairs of letters.

Tables 7 and 8 show mechanical properties for the Lys-containing films. In Table 7 we can observe that adequate control over TS, EB, and EM was achieved for FPH01, FPH05, and FPH06, all of which only had either ERL or ERS and no other polymer. The addition of HPMC was correlated with an increase in TS, decrease in EB, and a slight increase in EM (Table 7). This is indicative of less ductile yet more resistant films. The effect of HPMC over the mechanical properties of films is clearer after analysis of the derived mechanical parameters. TS/EM in an indicator of the level of internal stress in a film, the larger its value the higher the film crack resistance. RSE is also utilized to estimate crack resistance and is approximated from the surface energy of the film. Finally, TI is an estimation of energy absorbed per unit volume of film under stress. FPH01 is the formulation that possessed the largest TS/EM indicating high resistance to cracking (Table 8). The addition of HPMC reduced this value significantly except for FPH04; however, TS/EM values remained high and acceptable. In the same line, the RSE of the films increased with the increase of the content in HPMC, being highest for FPH04 at 10.32 N/mm2·%, indicating crack resistance. Comparison of TI indicates that except for FPH01 which resulted to be the toughest formulation, TI of all other formulations varies in acceptable ranges (Table 8).

TABLE 9
Differences among FPH series of formulations
based on the similarity factor, f₂.
Release profiles are similar if f₂ ≧ 50.

| f2 | FPH01 | FPH02 | FPH03 | FPH04 | FPH05 | FPH06 |
|---|---|---|---|---|---|---|
| FPH01 | — | 40.76 | 22.47 | 20.73 | 27.23 | 19.40 |
| FPH02 | | — | 34.46 | 31.64 | 18.11 | 12.65 |
| FPH03 | | | — | 56.79 | 9.75 | 5.89 |
| FPH04 | | | | — | 9.00 | 5.29 |
| FPH05 | | | | | — | 45.23 |
| FPH06 | | | | | | — |

From the drug release profiles shown in FIG. 21, we can observe an increase in the release rate and extent of release as the concentration of HPMC increased in the formulations (FPH01-FPH04). In accordance with the similarity value f2, FPH03 and FPH04 are the only formulations that render a similar Lys release profile. Therefore, an increase in the HPMC content from 30 to 50% w/w of polymer did not elicit significant differences in the release profile.

In Table 10, we can observe that except for FPH04, all formulations exhibit an anomalous release of Lys (from the Korsmeyer-Peppas equation). This is a consequence of systems that are water swellable, where drug release occurs by a combination of diffusion and case-II transport. In the case of FPH04, the release is more adequately modeled by the Higuchi model (evidenced by the higher R²). This indicates that drug release in this system follows Fickian diffusion through the polymer matrix. In addition, all formulations are better adjusted to the first order kinetics model (according to the R2). This model describes drug release from porous matrices, such as that formed in a water swollen polymethacrylate film, having a water soluble drug, such as the Lys-containing particles. From the release profile we can also observe that when Lys was added to the film formulation as a solid solution very little release was achieved over the 4 hour period of time. Molecules in solid solution are completely surrounded by the polymeric matrix and a higher number of interactions between polymer and Lys can be achieved. This results in a very slow release over the time period (below LOQ).

TABLE 10
Model parameters and adjusted R² values for the FPH series of formulations.

| | Korsmeyer-Peppas | | | Higuchi | | First order | | |
|---|---|---|---|---|---|---|---|---|
| | Q = k × tⁿ | | | Q = k × t^{0.5} | | Q = k × (1 − e^{-nt}) | | |
| Formulation | k | n | Adj R² | k | Adj R² | k | n | Adj R² |
| FPH01 | 0.1092 | 0.7702 | 0.9980 | 0.1407 | 0.9391 | 0.4956 | 0.2474 | 0.9995 |
| FPH02 | 0.1728 | 0.6287 | 0.9955 | 0.1943 | 0.9791 | 0.4702 | 0.4672 | 0.9965 |
| FPH03 | 0.2255 | 0.6604 | 0.9874 | 0.2612 | 0.9659 | 0.6579 | 0.4335 | 0.9998 |
| FPH04 | 0.2800 | 0.4875 | 0.9751 | 0.2769 | 0.9881 | 0.5340 | 0.8316 | 0.9966 |
| FPH05 | 0.0457 | 0.5837 | 0.9961 | 0.0492 | 0.9894 | 0.1114 | 0.5380 | 0.9911 |

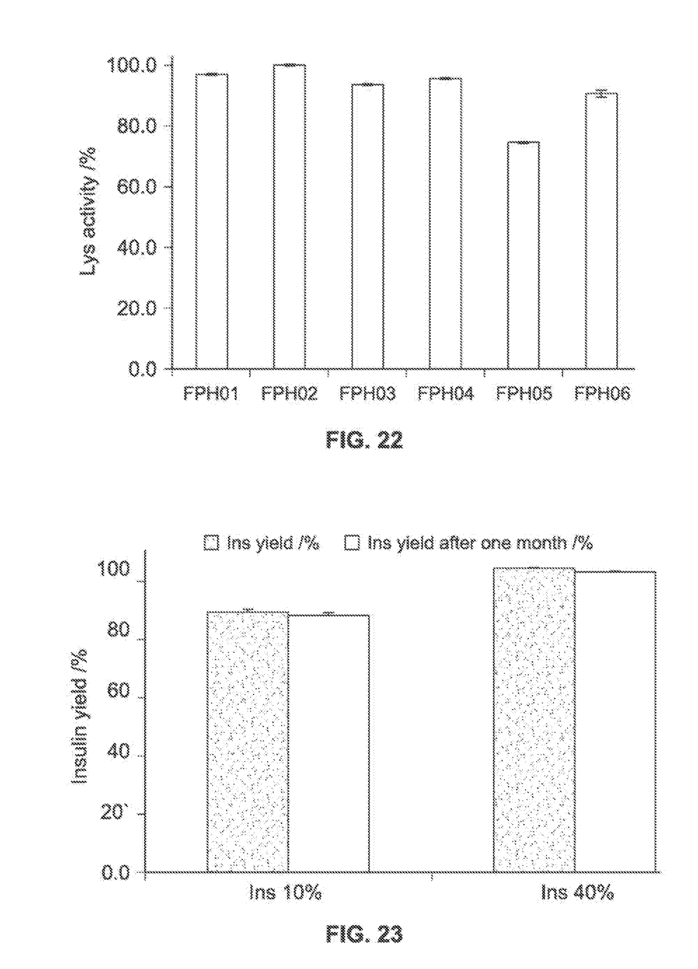

After release for 24 hours in dissolution media, the activity of the Lys released was evaluated to measure any decrease in activity as an indicator of enzyme stability. As depicted in FIG. 22, Lys remaining activity was excellent for all the formulations studied revealing that the processing of the manufactured particles into films for buccal delivery did not render the enzyme inactive.

### Example III

### Films for Buccal Delivery of Insulin-Coated Nanoparticles

Submicron sized and nanosized particles embedded in the polymer matrix have been found to produce films that comply with adequate mechanical and bioadhesive properties. Insulin-coated nanoparticles (ICNP) were manufactured and then embedded in film formulations for buccal Ins delivery and studied for physicochemical properties, release and permeation through a human buccal mucosa three dimensional model.

The manufacturing process was based on the antisolvent co-precipitation method. First, Val was dissolved in acid phthalate buffer pH 2.2 for a concentration of 61.2 mg/mL (or 90% w/v of its saturation concentration). Two different formulations were then manufactured to contain 10% and 40% w/w of Ins based in solid content. By means of an Aeroneb Pro® vibrating mesh nebulizer, the aqueous phase was then added to the antisolvent organic phase. A 0.008 mM Span 60 solution was utilized as a stabilizing surfactant. Finally, during the addition of the aqueous phase high energy mixing is provided by means of a Branson Sonifier 450 probe sonicator (Branson Ultrasonics, Danbury, Conn.). After addition of the total volume of aqueous phase sonication is maintained for a further 20 minutes.

Casting solutions were prepared by combining two organic solutions and cast overnight in PTFE molds. Acetone was used to dissolve or suspend the polymer combinations as depicted in Table 5. This solution was combined in a 4:6 acetone to IPA ratio with suitable amounts of slurries to yield the final casting solution. After 24 hours, films were peeled off and stored in aluminum foil sachets in a dessicator until characterization.

TABLE 11
Film formulations studied to investigate drug release and
uniformity of films containing Ins particles.

| | Eudragit RL | HPMC | TEC |
|---|---|---|---|
| Formulations | % w/w | % w/w | % w/w |
| ERL-Ins | 90 | — | 10 |
| ERL-HPMC-Ins | 45 | 45 | 10 |

We have successfully obtained Insulin-coated nanoparticles (ICNP) by an antisolvent precipitation process (Table 12). A high content of peptide resulted in the smallest particle size (323±8 nm) and highest ζ−Potential (32.4±0.8 mV).

TABLE 12
Particle size reported as z-average, polydispersity index, and ζ-potential
of the Ins formulations investigated. Results are represented
as the mean and standard deviation in parenthesis.

| | | Polidispersity | |
|---|---|---|---|
| Formulation | Z-average/nm | index* | ζ-Potential/mV |
| Ins 10% | 819 (48) | 0.44 (0.14) | 18.3 (0.3) |
| Ins 40% | 323 (8) | 0.42 (0.02) | 32.4 (0.8) |

*except for polydispersity indices, all pairwise comparison were significantly different (p < 0.001).

In our studies we found that the addition of Ins significantly decreased particle size from 888±10 nm for pure Val nanoparticles to 819±48 nm with 10% Ins and further down to 323±8 nm with 40% Ins.

The antisolvent co-precipitation process has also been described as a process to render highly active and stable protein and peptide-containing particles. Here we have found that after manufacture, even though Ins is subjected to high energy mixing via sonication, very high yields are achieved (FIG. 23). Furthermore, high stability of Ins content in the particles manufactured were found after one month of storage under room conditions.

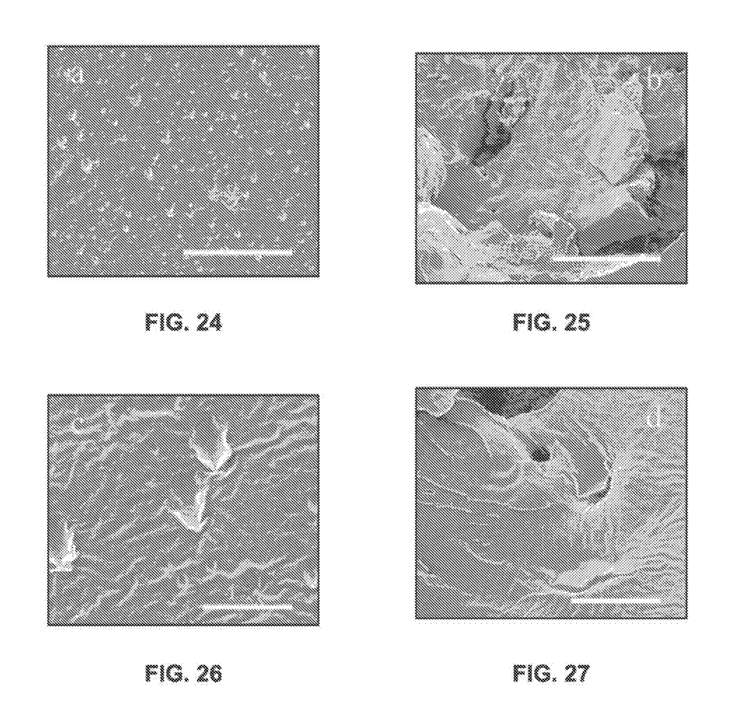

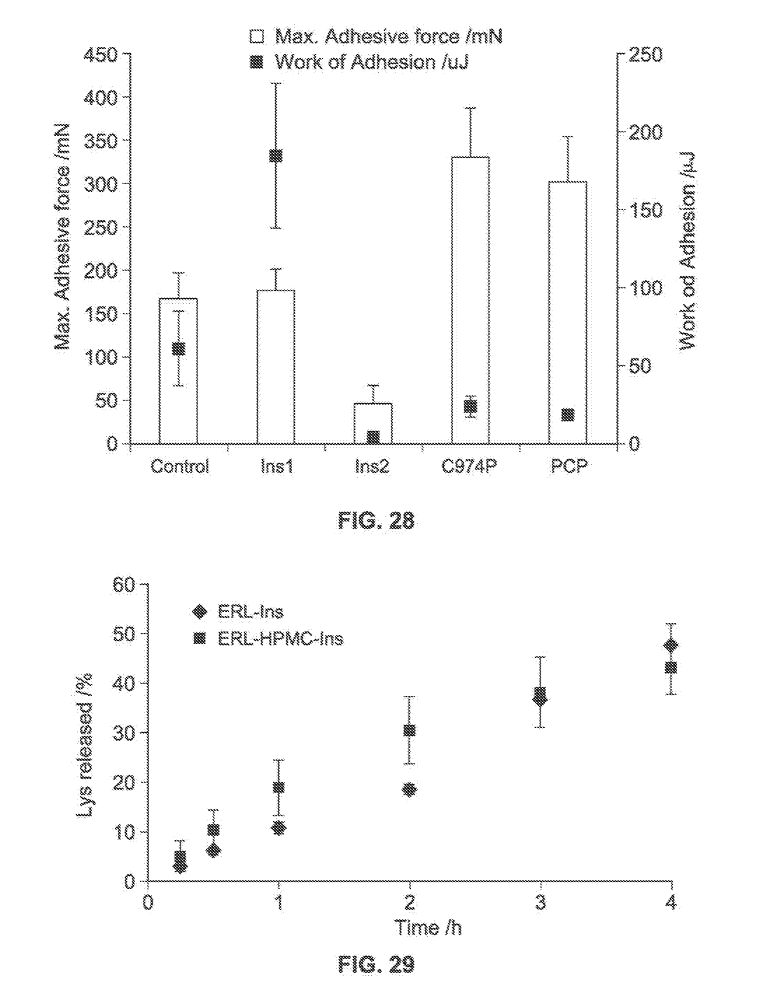

As shown in FIGS. 24-27, successful manufacture of films was achieved with both polymers utilized, ERL films appeared homogeneous to the eye and inspection of cross-sections obtained by freeze fracture revealed a homogeneous distributions of what appears to be flakes of Ins-coated nanoparticles (FIG. 26). Alternatively, films manufactured by combining ERL and HPMC present with a more discontinuous matrix due to the distribution of HPMC throughout ERL. However, distinct domains reveal the presence of similar flake-like particles in the polymer matrix, similarly to the evidence found in ERL films (FIG. 27).

Table 13 shows the direct and derived mechanical properties for Ins-containing films. In Table 13 we can observe that the inclusion of particles in the polymer matrix decreases strength and toughness, but increase elongation slightly compared to pure ERL films plasticized to the same extent with TEC. The disruption of the polymer continuum results in slightly softer acceptable films as solid dosage form. The addition of HPMC drastically decreased elongation and highly increased strength which is associated with a decrease in TI.

TABLE 13
Direct and derived mechanical properties for Ins-containing films.
Results are represented as the mean and standard deviation in parenthesis

| | Tensile strength | Elongation at break | Elastic modulus |
|---|---|---|---|
| Formulation | N/mm² | % | N/mm²/% |
| Control | 1.455 (0.120)* | 217.7 (30.5) | 0.513 (0.062) |
| Ins1 | 0.514 (0.010)* | 277.0 (14.3) | 0.099 (0.009) |
| Ins2 | 5.500 (0.945) | 14.6 (3.5) | 1.337 (0.275) |
| | TS:EM | Relative Surface | Toughness index |
| | %⁻¹ | Energy N/mm² · % | N/mm² · % |
| Control | 2.86 (0.34) | 2.09 (0.37)* | 212.69 (44.34) |
| Ins1 | 5.21 (0.52) | 1.34 (0.14)* | 94.83 (3.90)* |
| Ins2 | 4.14 (0.21) | 11.33 (1.65) | 52.68 (10.18)* |

*indicated pairs of data among parameters were not found to be statistically different (p > 0.05).

FIG. 29 reveals that both formulations presented with similar release profiles, controlling drug release to about 50% over a 4 hours period of time. Even though ERL-HPMC films exhibit faster release at earlier time points, the high variability obtained is responsible for the similarity factor (f2) to reach a value of 60.8 indicating no statistical difference between profiles. The high extent of variability can be attributed to the presence of HPMC which is responsible for an increase in heterogeneity of the polymer matrix. In our studies we observed an increase in variability due to the higher release of HPMC-rich domains that contribute to heterogeneity. Signs of heterogeneity can also be observed in the cross-sections obtained by freeze fracture in FIGS. 24-27.

Both release profiles are best explained by the Korsmeyer-Peppas equation by inspection of the R2 values obtained in Table 14.

TABLE 14
Model parameters and adjusted R² values for the insulin-containing films.

| | | | | | | First order | |
|---|---|---|---|---|---|---|---|
| | Korsmeyer-Peppas | | | Higuchi | | Q = 100 × | |
| Formu- | Q = k × tⁿ | | | Q = k × t^{0.5} | | (1 − e^{-nt}) | |
| lation | k | n | Adj R² | k | Adj R₂ | n | Adj R² |
| ERL- | 10.560 | 1.086 | 0.9885 | 19.059 | 0.8378 | 0.146 | 0.9705 |
| Ins | | | | | | | |
| ERL- | 18.299 | 0.662 | 0.9888 | 20.976 | 0.9605 | 0.163 | 0.9644 |
| HPMC- | | | | | | | |
| Ins | | | | | | | |

What is claimed is:

1. A bioadhesive drug delivery film comprising at least one polymer layer in which submicron-sized drug particles are releasably embedded.

2. The bioadhesive drug delivery film of claim 1 wherein the polymer layer in which the drug particles are releasably embedded comprises a bioadhesive polymer.

3. The bioadhesive drug delivery film of claim 1 comprising first and second polymer layers and wherein the drug particles are releasably embedded in the first layer and the second layer comprises a bioadhesive polymer.

4. The bioadhesive drug delivery film of claim 3 wherein the first polymer layer does not contain a bioadhesive polymer.

5. The drug delivery film of claim 1 further comprising a drug barrier coating at least a portion of the bioadhesive polymer so as to restrict the direction in which drug release can occur.

6. The drug delivery film of claims 1, 2, 3, 4, or 5 further comprising a releasably contained permeation enhancer.

7. The drug delivery film of claims 1, 2, 3, 4, or 5 wherein the permeation enhancer is releasably contained within the same polymer layer as the drug particles.

8. The drug delivery film of claim 1 wherein the polymer disintegrates over time.

9. The drug delivery film of claim 1 wherein the polymer releases the drug over an extended period of time.

10. The drug delivery film of claim 1 and 9 wherein the mechanism of drug release is controlled disintegration of the polymer.

11. The drug delivery film of claim 1 wherein the bioadhesive layer comprises a polymethacrylate-based polymer.

12. The drug delivery film of claim 11 wherein the bioadhesive layer comprises Eudragit RL.

13. The drug delivery film of claims 1, 2, 3, 4 or 5 wherein the drug particles are microparticles coated with the drug.

14. The drug delivery film of claim 1 further comprising a physical structure configured to enhance penetration of the drug.

15. The drug delivery film of claim 14 wherein the physical structure comprises microneedles.

16. The drug delivery film of claim 15 wherein the microneedles are biocompatible.

17. The drug delivery film of claim 15 or 16 wherein the microneedles are biodegradable.

18. A method for producing a bioadhesive drug delivery film comprising:
producing sub-micron sized biological-coated particles by:
providing a solvent containing a biological of interest and a core molecule; and
adding the solvent in a drop-wise manner to a miscible antisolvent under high energy mixing conditions so as to yield biological-coated submicron particles;
embedding the biological-coated particles in a polymer which may or may not be bioadhesive and;
if the polymer is not bioadhesive, adhering the non-bioadhesive polymer containing the embedded biological-coated particles to a bioadhesive polymer to produce a bioadhesive film.

19. The method of claim 18 further comprising adhering a drug barrier layer to a portion of the bioadhesive film.

20. The method of claim 18 or 19 wherein the step of adding the solvent in a drop-wise fashion is accomplished by way of a nebulizer.

21. The method of claim 18 or 19 wherein the high energy mixing conditions are achieved by way of a sonicator.

22. The method of claim 2 18 or 19 wherein the bioadhesive polymer comprises polymethacrylate.

23. The method of claim 18 or 19 further comprising adding a permeation enhancer to the bioadhesive or non-bioadhesive polymer.

24. A method of delivering a drug to a patient comprising adhering a drug-delivery film comprising at least one polymer layer in which submicron-sized drug particles are releasably embedded to a mucosal membrane, such that the submicron-sized particles are able to diffuse out of the polymer layer and enter the patient's circulatory system without passing through the patient's digestive system.
